(19) United States
(12) Patent Application Publication
(10) Pub. No.: US 2022/0093005 A1
(43) Pub. Date: Mar. 24, 2022

(54) Silicone Oil-Induced Ocular Hypertension Glaucoma Model

(71) Applicant: The Board of Trustees of the Leland Stanford Junior University, Stanford, CA (US)

(72) Inventors: Yang Hu, Palo Alto, CA (US); Jie Zhang, Wuhan (CN)

(21) Appl. No.: 17/420,887

(22) PCT Filed: Jan. 16, 2020
(86) PCT No.: PCT/US2020/013958

§ 371 Date: Jul. 6, 2021

Related U.S. Application Data
(60) Provisional application No. 62/795,234, filed on Jan. 22, 2019.

(51) Int. Cl. G09B 23/30; A61F 9/007

(52) U.S. Cl. CPC G09B 23/306; A61F 9/00781

(57) ABSTRACT

Injection of silicon oil (SO) to the anterior chamber of an eye efficiently induces intraocular pressure (IOP) elevation. This effect occurs without causing overt ocular structural damage or inflammatory responses while simulating acute glaucomatous changes that human patients develop over years by inducing progressive RGC and ON degeneration and visual functional deficits within weeks. The anterior segments of the experimental eyes are not substantially affected, leaving clear ocular elements that allow easy and reliable assessment of in vivo visual function and morphology. More importantly, this is the only reversible ocular hypertension model by removing SO from the anterior chamber and particularly useful for testing neuro-protection treatment together with lowering IOP treatment. In summary, the acute ocular hypertension glaucoma model replicates secondary post-operative glaucoma. It is straightforward and reversible, does not require special equipment or repeat injections, and may be applicable to a range of animal species with only minor modifications.

### FIELD OF THE INVENTION

This invention relates to methods, devices and systems for the treatment of glaucoma.

### BACKGROUND OF THE INVENTION

Glaucoma is the most common cause of irreversible blindness and will affect more than 100 million individuals between 40 and 80 years of age by 2040. Annual direct medical costs to treat this disease in 2 million patients in the United States totaled $2.9 billion. Glaucoma is a neurodegenerative disease characterized by injury to the axons of retinal ganglion cells (RGCs) followed by progressive degeneration of RGC somata and axons within the retina and Wallerian degeneration of the myelinated axons in the optic nerve (ON). The level of intraocular pressure (IOP) is the most common risk factor. Current clinical therapies target reduction of TOP to retard glaucomatous neurodegeneration, but neuroprotectants are critically needed to prevent degeneration of RGCs and ON. Similar to other chronic neurodegenerative diseases, the search for neuroprotectants to treat glaucoma continues. To longitudinally assess the molecular mechanisms of glaucomatous degeneration and the efficacy of neuroprotectants, a reliable, reproducible, and inducible experimental ocular hypertension/glaucoma model is essential. Rodents serve as a mammalian experimental species of choice for modeling human diseases and large-scale genetic manipulations. Various rodent ocular hypertension models have been developed including spontaneous mutant or transgenic mice and rats and mice with inducible blockage of aqueous humor outflow from the trabecular meshwork (TM). While genetic mouse models are valuable to understand the roles of a specific gene in TOP elevation and/or glaucomatous neurodegeneration, the pathologic effects may take months to years to manifest. Inducible ocular hypertension that develops more quickly and is more severe term would be preferable for experimental manipulation and general mechanism studies, especially for neuroprotectant screening. Injection of hypertonic saline and laser photocoagulation of the episcleral veins and TM are commonly used in rats and larger animals. Although similar techniques also produce ocular hypertension in mice, they are technically challenging, and irreversible ocular tissue damage and intraocular inflammation complicate their interpretation. Intracameral injection of microbeads to occlude aqueous humor circulation through TM produces excellent TOP elevation and glaucomatous neurodegeneration. However, retaining microbeads at is the angle of the anterior chamber and controlling the degree of aqueous outflow blockade are difficult. Furthermore, its lengthy duration (6-12 weeks after microbeads injection) causes death of only less than 30% of RGC, leaving a narrow window for preclinical testing of neuroprotective therapies. It is therefore critically important to develop an effective ocular hypertension model that closely resembles human glaucoma, and that can be readily adapted among different species with minimal confounding factors.

Secondary glaucoma with acutely elevated TOP occurs as a post-operative complication following the intravitreal use of silicone oil (SO) in human vitreoretinal surgery. SO is used as a tamponade in retinal detachment repair because of its buoyancy and high surface tension. However, SO is lighter than the aqueous and vitreous fluids and an excess can physically occlude the pupil, which prevents aqueous flow into the anterior chamber. This obstruction increases aqueous pressure in the posterior chamber and displace the iris anteriorly, which causes angle-closure, blockage of aqueous outflow through TM, and a further increase in IOP. Prophylactic peripheral iridotomy that maintains the circulation between anterior and posterior chambers normally prevents this type of secondary glaucoma.

### SUMMARY OF THE INVENTION

A reliable glaucoma model that closely mimics the disease in humans is a prerequisite for studies of pathogenetic mechanisms and for selecting efficient neuroprotective treatments for clinical use. In the present invention, such a highly effective and reproducible model and method was developed. Injection of SO to the mouse anterior chamber efficiently induces a series of reactions, including pupillary block, blockage of the aqueous humor outflow from anterior chamber, is accumulation of aqueous humor in the posterior chamber, closure of the anterior chamber angle, and IOP elevation. These reactions occur without causing overt ocular structural damage or inflammatory responses while simulating acute glaucomatous changes that human patients develop over years by inducing progressive RGC and ON degeneration and visual functional deficits within weeks.

SO injection is limited to one eye (experimental eye) in each mouse, with the other eye (contralateral eye) receiving an equivalent volume of normal saline. This serves as a convenient internal control for the surgical procedure and for studies of RGC morphology and function. It is reasonable to conclude that TOP is elevated in the SOHU eyes because of impeded inflow and accumulation of aqueous humor in the posterior segment of the eye, rather than by an aspect of the surgical procedure, such as the cornea wound or inflammation, which was rare.

Because of the unique feature of pupillary block associated with SOHU, the TOP is elevated in the posterior part of the eye, but not in the anterior chamber. The inventors postulated that, after the pupil is sealed by SO, the large mouse lens, together with the iris and ciliary body, forms a rigid barrier that essentially disconnects the anterior and posterior chambers and thus shields the io anterior chamber from the high pressure in the posterior chamber. This pathogenesis gives the model two advantageous characteristics: 1) The anterior segments of the experimental eyes are not substantially affected, leaving clear ocular elements that allow easy and reliable assessment of in vivo visual function and morphology; 2) The high TOP of the posterior chamber causes pronounced glaucomatous neurodegeneration within 5-8 weeks, which facilitates testing neuroprotectants by allowing any benefit to be detected in a short period of experimental time.

Understanding the molecular mechanism of glaucoma and development of neuroprotectants are significantly hindered by the lack of a reliable animal model that accurately recapitulates human glaucoma. In this invention, we developed a model for the secondary glaucoma that is often observed in humans after silicone oil (SO) blocks the pupil or migrates into the anterior chamber following vitreoretinal surgery. We observed similar intraocular pressure (TOP) elevation after intracameral injection into mouse eyes of SO, and removing the SO allows the TOP level to quickly return to normal. This inducible and reversible model showed dynamic changes of visual function that correlate with progressive RGC loss and axon degeneration. We also used a single AAV vector for the first time to co-express miRNA-based shRNA and a neuroprotective transgene and further validated this model as an effective in vivo means to test neuroprotective therapies by targeting neuronal endoplasmic reticulum stress.

Embodiments of this invention and model can be adapted to other experimental animal species to produce stable, robust TOP elevation and significant neurodegeneration. The model produces standardized ocular hypertension-induced pathology and supports studies of pathogenetic o mechanisms and of selection of neuroprotectants for glaucoma.

The invention is embodiment as a model or device as a silicone oil-induced ocular hypertension glaucoma model distinguishing an experimental eye with an anterior chamber having in the anterior chamber a silicone oil droplet larger than 1.5 mm in diameter. The silicone oil droplet is equivalent to about 1-2 microliters. The model could be enhanced by a contralateral eye with an anterior chamber having in the anterior chamber a volume of saline which is used as a control eye relative to the experimental eye. The volume of saline is equivalent to about 1-2 microliters.

The invention is embodiment as a method of modeling intraocular hypertension distinguishing the steps of injecting into an anterior chamber of an experimental eye a silicone oil to form a droplet of at least 1.5 mm in diameter inside the anterior chamber. The injected silicone oil is equivalent to about 1-2 microliters. The method could further distinguish injecting into an anterior chamber of a contralateral eye a volume of saline which is used as a control eye relative to the experimental eye. The volume of saline is equivalent to about 1-2 microliters.

In one embodiment, the model is based on an animal, and it this teaching, specifically, a mouse model was used, however, the particular animal model is not limited to mice as it could also be a primate model or any other animal model that closely mimics the human eye anatomy and physiology.

### BRIEF DESCRIPTION OF THE DRAWINGS

FIG. 1A shows according to an exemplary embodiment of the invention silicone oil-induced ocular hypertension under-detected (SOHU) mouse model. SO intracameral injection, pupillary block, closure of the anterior chamber angle, and reopening of the angle of anterior chamber after pupil dilation.

FIG. 1B shows according to an exemplary embodiment of the invention silicone oil-induced is ocular hypertension under-detected (SOHU) mouse model. Representative anterior chamber OCT images of SOHU eyes in living animals showing the relative size of SO droplet to pupil and the corresponding closure or opening of the anterior chamber angle before and after pupil dilation. Curved arrow indicates the direction of aqueous humor flow.

FIG. 1C shows according to an exemplary embodiment of the invention silicone oil-induced ocular hypertension under-detected (SOHU) mouse model. Longitudinal TOP measurements at different time points before and after SO injection, and continuous measurements for 18 min after anesthesia with isoflurane at each time point.

FIG. 1D shows according to an exemplary embodiment of the invention silicone oil-induced ocular hypertension under-detected (SOHU) mouse model. The sizes of SO droplet and corresponding IOP measurements at different time points after SO injection; IOP measured 12-15 min after anesthesia. SO: SO injected eyes; CL: contralateral control eyes. Data are presented as means±s.e.m, SO >1.5 mm, n=17; SO≤1.5 mm, n=6.

FIG. 2A shows according to an exemplary embodiment of the invention dynamic changes in RGC morphology and visual function in living SOHU animals. Representative OCT images of mouse retina; circle indicates the OCT scan area surrounding ON head. GCC: ganglion cell complex, including RNFL, GCL and IPL layers; indicated by double end arrows.

FIG. 2B shows according to an exemplary embodiment of the invention dynamic changes in RGC morphology and visual function in living SOHU animals. Quantification of

GCC thickness, represented as percentage of GCC thickness in the SO eyes, compared to the CL eyes. n=10-20.

FIG. 2C shows according to an exemplary embodiment of the invention dynamic changes in RGC morphology and visual function in living SOHU animals. Visual acuity measured by OKR, represented as percentage of visual acuity in the SO eyes, compared to the CL eyes. n=10-20.

FIG. 2D shows according to an exemplary embodiment of the invention dynamic changes in RGC morphology and visual function in living SOHU animals. Representative waveforms of PERG in the contralateral control (CL) and the SO injected (SO identified with 2D_SO) eyes at different time points after SO injection. P1: the first positive peak after the pattern stimulus; N2: the second negative peak after the pattern stimulus.

FIG. 2E shows according to an exemplary embodiment of the invention dynamic changes in RGC morphology and visual function in living SOHU animals. Quantification of P1-N2 N2 amplitude, represented as percentage of P1-N2 amplitude in the SO eyes, compared to the CL eyes. n=13-15. Data are presented as means ±s.e.m, *: p<0.05, **: p<0.01, ***: p<0.001, ****: p<0.0001, one-way ANOVA with Tukey's multiple comparison test.

FIG. 3A shows according to an exemplary embodiment of the invention glaucomatous RGC soma and axon degeneration in SOHU eyes. Upper panel, confocal images of whole flat-mounted retinas showing surviving RBPMS-positive RGCs at different time points after SO injection. Scale bar, 100 mm. Middle panel, confocal images of a portion of flat mounted retinas showing surviving RBPMS-positive RGCs at different time points after SO injection. Scale bar, 20 mm. Lower panel, light microscope images of semi-thin transverse sections of ON stained with PPD at different time points after SO injection. Scale bar, 10 mm.

FIGS. 3B-C show according to an exemplary embodiment of the invention glaucomatous RGC soma and axon degeneration in SOHU eyes. Quantification of surviving RGCs in the peripheral retina (n=11-13) and surviving axons in ON (n=10-16) at different time points after SO injection, represented as percentage of SO eyes compared to CL eyes. Data are presented as means ±s.e.m. *p<0.05, **p<0.01, ***: p<0.001, ****: p<0.0001; one-way ANOVA with Tukey's multiple comparison test.

FIG. 4A shows according to an exemplary embodiment of the invention SO itself does not cause glaucomatous degeneration. TOP measurements at different time points after intravitreal SO injection. n=15.

FIG. 4B shows according to an exemplary embodiment of the invention SO itself does not cause glaucomatous degeneration. Visual acuity measured by OKR, represented as percentage of visual acuity in the SO eyes, compared to the CL eyes. n=13-15.

FIG. 4C shows according to an exemplary embodiment of the invention SO itself does not cause glaucomatous degeneration. Quantification of P1-N2 amplitude of PERG, represented as percentage of P1-N2 amplitude in the SO eyes, compared to the CL eyes. n=12-15.

FIG. 4D shows according to an exemplary embodiment of the invention SO itself does not cause glaucomatous degeneration. Quantification of GCC thickness measured by OCT, represented as percentage of GCC thickness in the SO eyes, compared to the CL eyes. n=11-13.

FIG. 4E shows according to an exemplary embodiment of the invention SO itself does not cause glaucomatous degeneration. Upper panel, confocal images of portions of flat-mounted retinas showing surviving RBPMS-positive RGCs at 8wpi after intravitreal SO injection and contralateral naive eye. Scale bar, 20 mm. Lower panel, light microscope images of semi-thin transverse sections of ON stained with PPD at 8wpi after intravitreal SO injection and contralateral naive eye. Scale bar, 10 mm.

FIG. 4F shows according to an exemplary embodiment of the invention SO itself does not cause glaucomatous degeneration. Quantification of surviving RGCs (n=10) and surviving axons in ON (n=10) at 8wpi after intravitreal SO injection, represented as percentage of SO eyes compared to the CL eyes. Data are presented as means ±s.e.m, Student t-test.

FIG. 4G shows according to an exemplary embodiment of the invention SO itself does not cause glaucomatous degeneration. Upper panel, confocal images of portion of flat-mounted retinas showing surviving RBPMS positive RGCs at 8wpi after intracameral SO injection (small size of SO droplet, ≤1.5 mm) and contralateral naive eye. Scale bar, 20 mm. Lower panel, light microscope images of semi-thin transverse sections of ON stained with PPD at 8wpi after intracameral SO injection and contralateral naive eye. Scale bar, 10 mm.

FIG. 411 shows according to an exemplary embodiment of the invention SO itself does not cause glaucomatous degeneration. Quantification of surviving RGCs (n=12) and surviving axons in ON (n=13) at 8wpi, represented as percentage of SO eyes compared to the CL eyes. Data are presented as means ±s.e.m, Student t-test.

FIG. 5A shows according to an exemplary embodiment of the invention. SOHU is reversible by SO removal. Representative images of SOHU eyes before and after SO removal, and anterior chamber OCT images in living animals showing the relative size of SO droplet to pupil and the corresponding closure or opening of the anterior chamber angle before and after SO removal.

FIG. 5B shows according to an exemplary embodiment of the invention. SOHU is reversible by SO removal. TOP measurements before and after SO removal at different time points. n=16.

### DETAILED DESCRIPTION

The present invention is a method and model developed as a procedure for intracameral injection of silicone oil (SO) to block the pupil, which causes acute ocular hypertension and significant retinal ganglion cell (RGC) and optical nerve (ON) degeneration. The present invention demonstrates that embodiments of this invention, which may be adaptable to different species, induces stable intraocular pressure (TOP) elevation and profound neuronal response to ocular hypertension in the retina that will expedite selection of neuroprotectants and establishing the pathogenesis of acute ocular hypertension-induced glaucoma. First, the materials and methods will be discussed, after which results of using the method and model will be presented. For the io purpose of model development and testing of the methodology a mice model was used.

METHODS

The following description is an embodiment of the model as a detailed protocol for SO-induced ocular hypertension in a mouse eye, including SO injection and removal and TOP measurement.

Mice

C57BL/6J WT mice were purchased from Jackson Laboratories (Bar Harbor, Me.).

Ocular Hypertension Induction by Intracameral Injection of SO
Prepare a glass micropipette for intracameral SO injection by pulling a glass capillary with a pipette puller to generate a micropipette. Cut an opening at the tip of the micropipette and further sharpen the tip with a microgrinder-beveling machine to make a 35°-40° bevel.
Polish the edges of the bevel and remove all debris by washing with water. Autoclave the micropipette before use.
Prepare the paracentesis needle for the corneal entry. To do so, attach a 32 G needle to a 5 mL syringe on a Luer lock, and further secure it with tape. Bend the needle bevel tip face up at 30°.
Prepare the SO injector by attaching and securing a blunt end 18 G needle on a 10 mL syringe first. Then attach a plastic tube with the 18 G needle on one end and fill up with SO as needed through the other end.
Attach the sterilized micropipette to the plastic tube and push the syringe plunger to fill the entire micropipette with SO.

Intracameral SO Injection for One Eye
Place a 9-10-week-old male C57B6/J mouse into an induction chamber with 3% isoflurane mixed with oxygen at 2 L/min for 3 min.
Intraperitoneally inject 2,2,2-tribromoethanol at 0.3 mg/g body weight. NOTE: Unlike ketamine/xylazine, 2,2,2-tribromoethanol does not cause obvious pupil dilation.
Check for the lack of response to a toe pinch and the lack of movement of the whiskers or the tail to determine the anesthetic strength.
Place the mouse in a lateral position on a surgery platform. To reduce its sensitivity during the procedure, apply one drop of 0.5% proparacaine hydrochloride to the cornea before the injection.
Make an entry incision with the 32 G paracentesis needle at the superotemporal quadrant, about 0.5 mm from the limbus.
Tunnel through the layers of the cornea for about 0.3 mm before piercing into the anterior chamber. Be careful not to touch the lens or iris.
Withdraw the needle slowly to release some aqueous humor (about 1-2 μL) from the anterior chamber through the tunnel (paracentesis).
Wait ˜8 min to further decrease the TOP. This can be determined by measuring the contralateral, control eye.
Insert the glass micropipette preloaded with SO through the corneal tunnel into the anterior chamber, with the bevel facing down to the iris surface.
Push the syringe plunger slowly to inject SO into the anterior chamber until the SO droplet covers most of the iris surface, ˜2.3-2.4 mm in diameter.
Leave the micropipette in the anterior chamber for 10 s more before withdrawing it slowly.
Gently push the upper eyelid to close the cornea incision to minimize SO leakage.
Apply antibiotic ointment (bacitracin-neomycin-polymyxin) to the eye surface.
Throughout the procedure, frequently moisten the cornea with artificial tears.
Keep the mouse on the heating pad until fully recovered from anesthesia.

SO Removal
Prepare the irrigation system.
   Prepare the irrigating solution according to the manufacturer's instructions and place it in the irrigation bottle. Elevate the irrigating solution bottle to 110-120 cm (81-88 mmHg) above the surgery platform.
   Attach an IV administration set to the irrigating solution bottle. Remove air bubbles from the IV tubing. Connect a 33 G needle bent to 20° face up to the IV tubing.
To prepare the drainage system, remove the plunger from a 1 mL syringe. Attach a 33 G needle to the syringe and bend the needle to 20°.
Remove SO from the anterior chamber.
   Intraperitoneally inject 2,2,2-tribromoethanol (0.3 mg/g body weight). Check for the lack of response to the toe pinch to determine the anesthetic strength and the lack of movement of the whiskers or the tail.
   Place the mouse on a surgery platform and secure it in the lateral position with tape. Apply one drop of 0.5% proparacaine hydrochloride to the cornea to reduce its sensitivity.
   Make two incisions in the temporal quadrant of the cornea between ˜2 and 5 o'clock at the edge of the SO droplet using the premade 32 G paracentesis needle.
   Insert a 33 G irrigation needle connected to irrigating solution through one corneal incision, maximum speed.
   Insert another 33 G drainage needle attached to the syringe without a plunger through the other corneal incision to allow the SO droplet to exit the anterior chamber while irrigating with irrigating solution.
   Withdraw the drainage needle, then the irrigation needle.
   Inject an air bubble into the anterior chamber to maintain its normal depth and press to close the corneal incision.
   Apply antibiotic ointment to both eyes.
   Keep the mouse on the heating recovery pad until fully recovered from the anesthesia.

IOP Measurement Once a Week
Place the mouse into an induction chamber perfused with 3% isoflurane mixed with oxygen at 2L/min for 3 min.
Intraperitoneally inject xylazine and ketamine (0.01 mg xylazine/g, 0.08 mg ketamine/g).
Keep the cornea moist by applying artificial tears throughout the procedure.
Wait about 15 min to allow the pupil to fully dilate.
Measure the TOP of both eyes using a tonometer according to product instructions. Bring the tonometer near the mouse eye. Keep the distance from the tip of the probe to the mouse cornea at about 3-4 mm. Press the measuring button 6x to generate one reading. Three machine-generated readings are obtained from each eye to acquire the mean TOP.
Sacrifice the animals at 8 weeks after SO injection and perform immunohistochemistry of whole-mount retina, RGC counting, optic nerve (ON) semi-thin sections, and quantification of surviving axons.

RESULTS

Intracameral SO Injection Induces Ocular Hypertension by Blocking the Pupil and Aqueous Humor Drainage

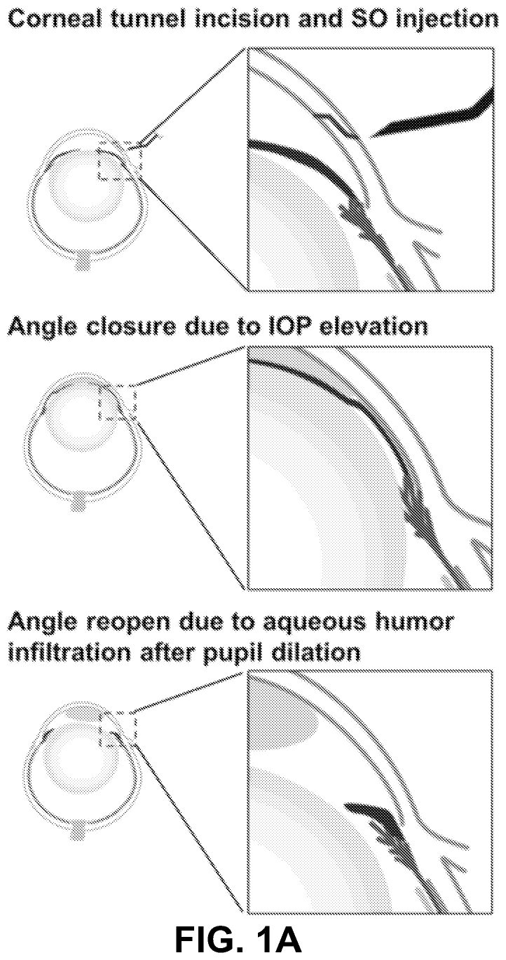

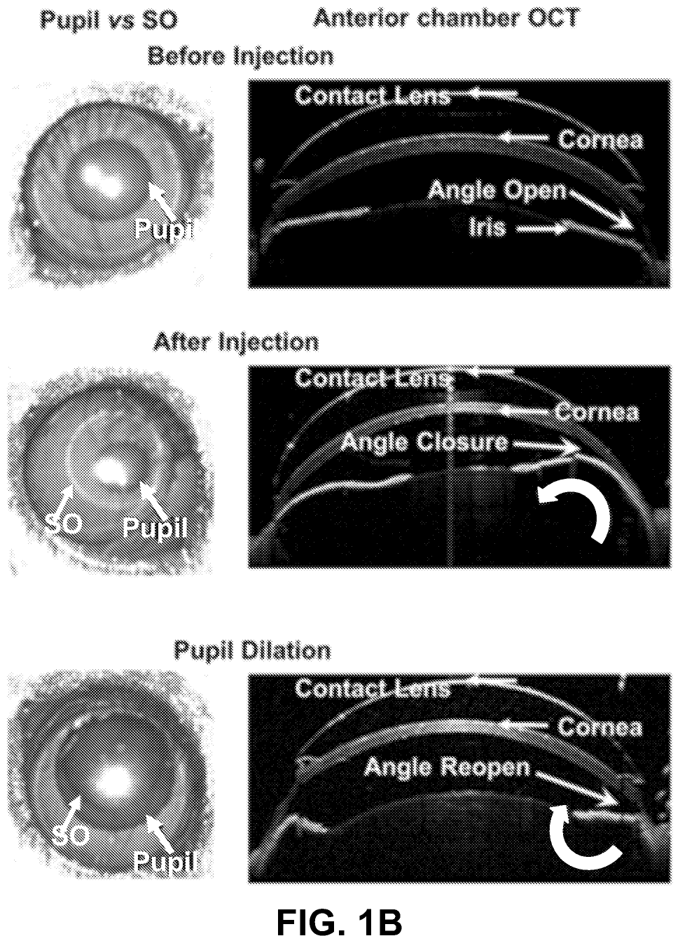

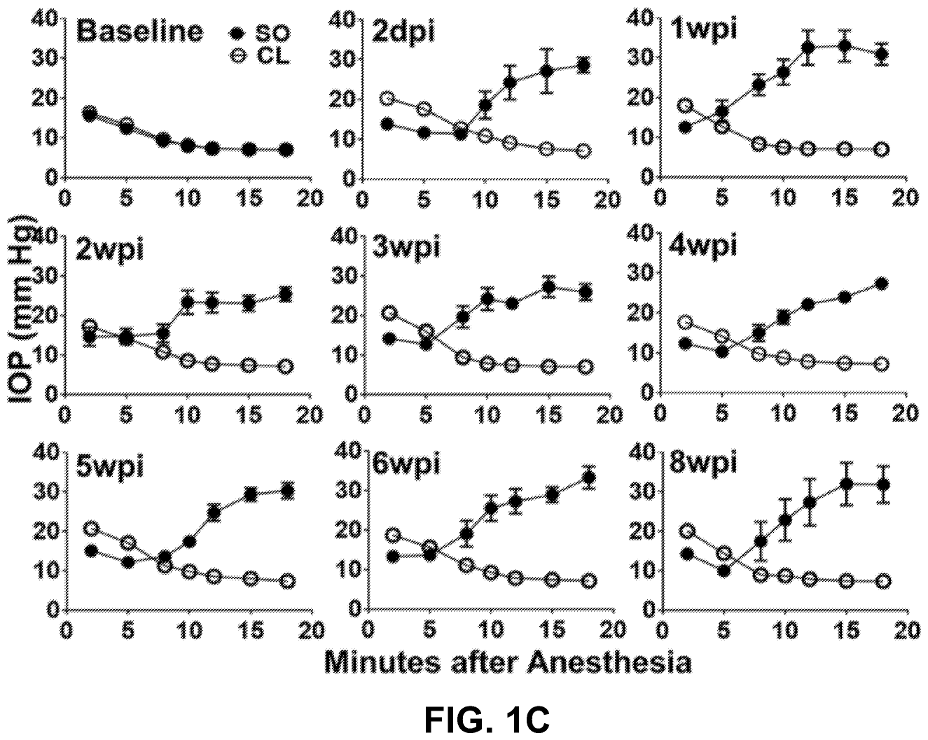

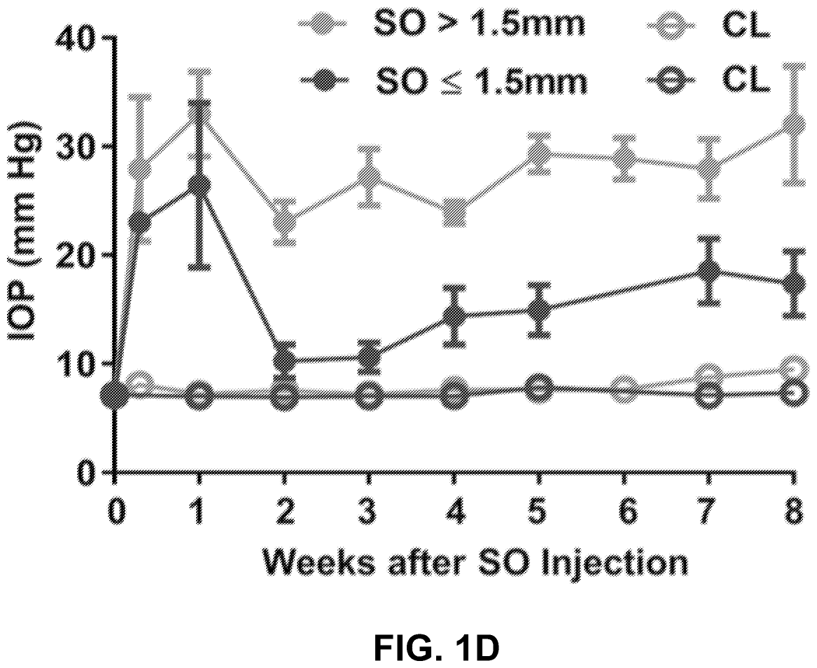

Although intravitreal injection of SO in vitreoretinal surgeries can cause post-operative secondary glaucoma in humans, the inventors reasoned that direct injection of SO into the anterior chamber of mice would be more efficient, preventing the need to remove the vitreous and reducing toxicity due to direct contact with the retina. As shown in FIGS. 1A, B, after intracameral injection SO forms a droplet in the anterior chamber that contacts the surface of the iris and tightly seals the pupil due to high surface tension. To test whether SO blocks migration of liquid from the back of io the eye to the anterior chamber, dye (DiI) was injected into the posterior chamber and visualized its migration into the anterior chamber. In dramatic contrast to a normal naive eye, in which copious dye passed through the pupil and appeared in the anterior chamber almost immediately after injection, no injected dye reached the anterior chamber of the SO eye. This result indicates that SO causes effective pupillary block.

The ciliary body constantly produces aqueous humor, which accumulates in the posterior chamber and pushes the iris forward. When the iris root touches the posterior corneal surface, the anterior chamber angle closes (FIG. IA), as evidenced by live anterior chamber optical coherence tomography (OCT) (FIG. 1B). The angle closure can further impede the outflow of aqueous humor through TM and may also contributes to TOP elevation. Dilation of the pupil until it is larger than the SO droplet can relieve the pupillary block. It was shown that after pupil dilation aqueous humor floods into the anterior chamber and pushes the SO droplet away from the iris, which reopens the anterior chamber angle (FIGS. 1A, B). Together, these results characterize the series of reactions initiated by intracameral SO injection, including the physical mechanisms of SO-induced pupillary block, posterior accumulation of aqueous humor, peripheral angle-closure, and IOP elevation.

The IOP was measured of the experimental eyes once weekly for 8 weeks after a single SO injection and the contralateral control (CL) eyes after a single normal saline injection. Surprisingly, IOP was lower in the SO eyes than in CL eyes when measured immediately after anesthetizing the animals with isoflurane (FIG. 1C). The TonoLab tonometer used to measure mouse IOP is based on a rebound measuring principle that uses a very light weight probe to make momentary contact with the center of the cornea, which primarily measures the pressure of anterior chamber. Measurements over extended periods of time showed the IOP of the SO eyes to be progressively and significantly elevated, in dramatic contrast to the CL eyes, in which IOP decreased over time. The increasing IOP in the SO eyes closely correlated with the change in pupillary size, indicating a significant role of pupillary block. Pupillary dilation removed the pupillary block and allowed the tonometer to detect higher IOP after aqueous humor migration into the anterior chamber, which reflects the elevated IOP in the posterior segment of the eye. Pupillary size reached its maximum and IOP reached to its plateau about 12-15 min after induction of anesthesia with continuous isoflurane inhalation. In mice in which the IOP was measured for as long as 30 min under anesthesia, however, the IOP eventually declined, indicating effective TM clearance of aqueous during this time (not shown). Therefore, the time period (12-15 min after induction of anesthesia) for measuring IOP was standardized in later experiments. Because the unique feature of this novel experimental glaucoma model is that the ocular hypertension is under-detected in non-dilated eyes, this was named ‘SO-induced ocular hypertension under-detected (SOHU)”.

IOP elevation in the SO eye started as early as 2 days post injection (2 dpi) and remained stable for at least 8 weeks (the longest time point tested) at an IOP about 2.5-fold that of CL eyes, if the diameter of the SO droplet was larger than 1.5 mm (FIG. 1D). This size of SO droplet was achieved in about 80% of mice, but in the 20% of mice with a small SO droplet (≤1.5 mm) in the anterior chamber due to poor injection or oil leaking, in which the IOP initially increased but dropped soon afterwards (FIG. 1D). Therefore, by observing the size of the SO droplet, it is io convenient to identify mice very early that would not show elevated IOP and exclude them from subsequent experiments.

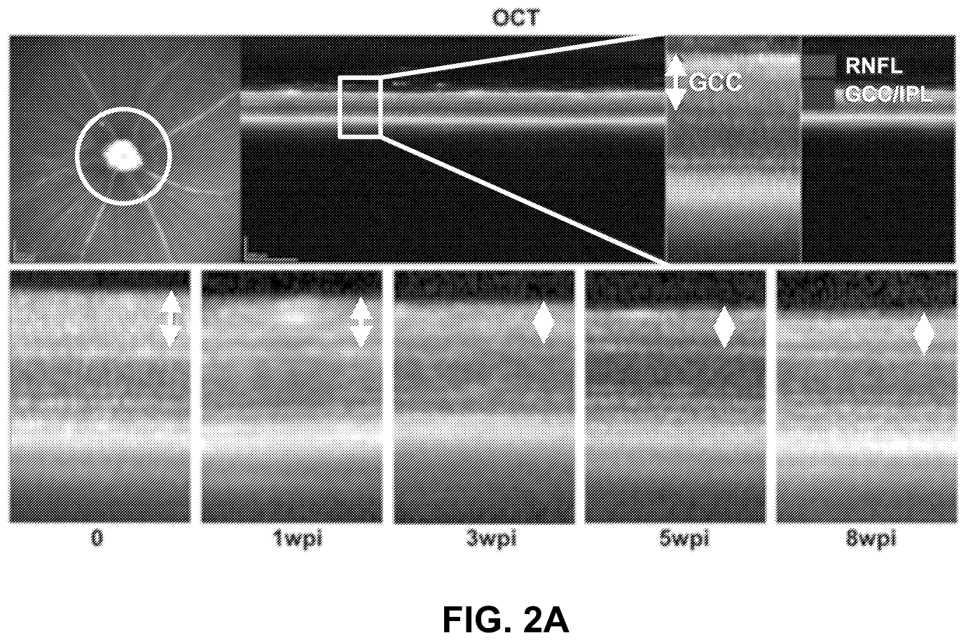

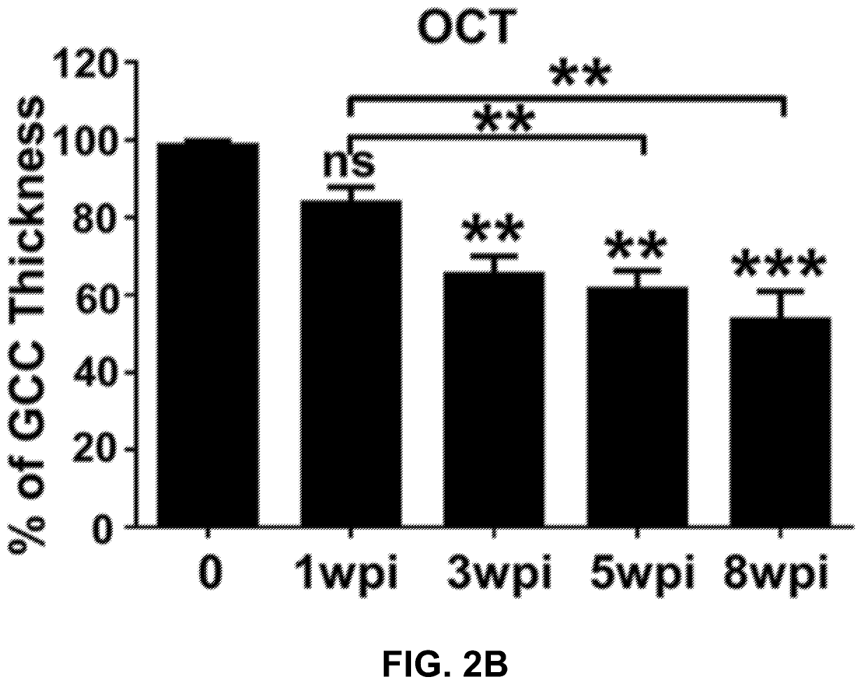

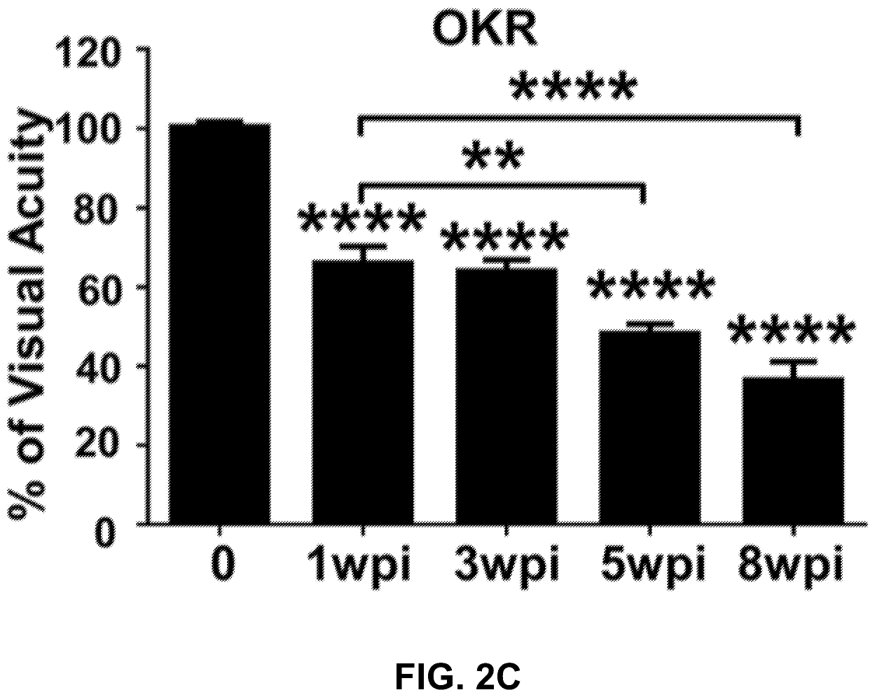

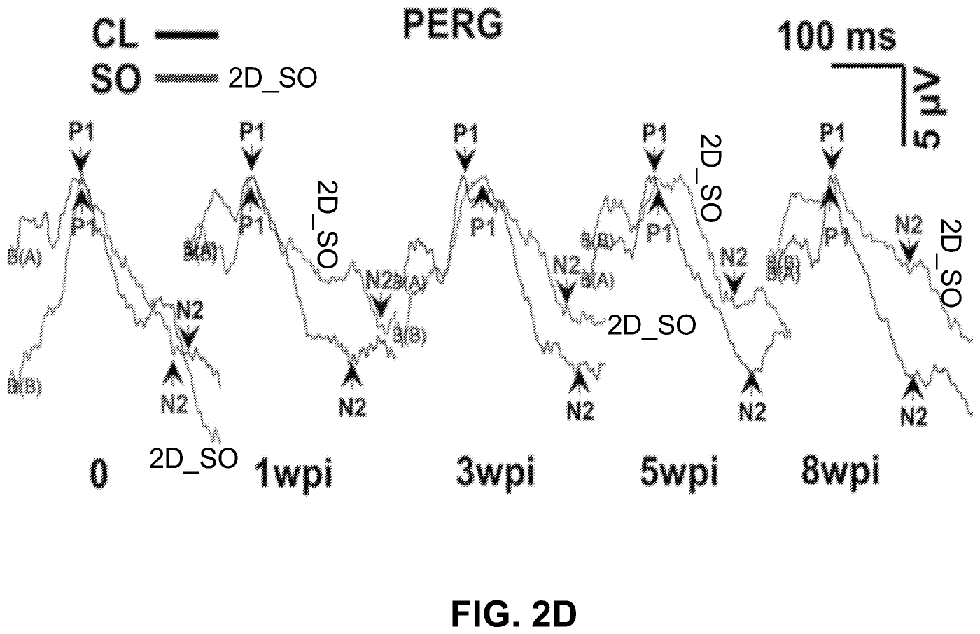

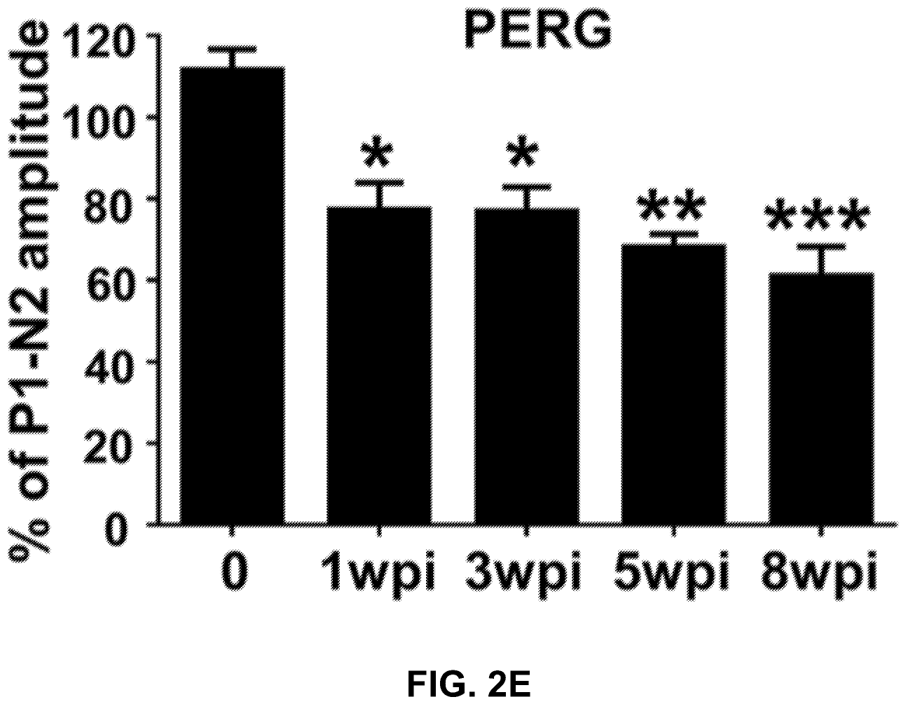

Visual function deficits and dynamic morphological changes in SOHU eyes of living animals To determine the dynamic changes in RGC morphology and function in SOHU eyes, the thickness of the ganglion cell complex (GCC) was longitudinally measured by OCT, visual acuity by the optokinetic tracking response (OKR), and general RGC function by pattern electroretinogram (PERG) in living animals. Clinically, the thickness of the retinal nerve fiber layer (RNFL) measured by posterior OCT serves as a reliable biomarker for glaucomatous RGC degeneration. Because the mouse RNFL is too thin to be reliably measured, the thickness of GCC was used, including RNFL, ganglion cell layer (GCL) and inner plexiform layer (IPL) together, to monitor degeneration of RGC axons, somata, and dendrites caused by ocular hypertension. GCC in SOHU eyes became gradually and progressively thinner (about 84%, 65%, 61% and 53% of CL eyes) at 1, 3, 5, and 8 weeks post injection (wpi). GCC thinning is statistically significant at 5 and 8 wpi compared to 1 wpi (FIGS. 2A, B). These results indicate progressive RGC degeneration in response to IOP elevation in SOHU eyes.

OKR is a natural reflex that objectively assesses mouse visual acuity. The mouse eye will only track a grating stimulus that is moving from the temporal to nasal visual field, which allows both eyes to be measured independently. It has been used to establish correlations between visual deficit and RGC loss in the DBA/2 glaucoma mouse model. The visual acuity of SOHU eyes decreased rapidly at lwpi, which may due to the presence of SO in the anterior chamber. However, o the further decreased visual acuity at 5 and 8 wpi compared to 1 wpi indicates progressive visual function deficits in the SOHU eyes (FIG. 2C). PERG is an important electrophysiological assessment of general RGC function, in which the ERG responses are stimulated with contrast-reversing horizontal bars alternating at constant mean luminance. The PERG system measured both eyes at the same time, so there was an internal control to use as a reference and normalization to minimize the variations. Consistent with visual acuity deficit, the P1-N2 amplitude ratio of the SO eyes to CL eyes decreased significantly (FIGS. 2D, E). However, that the lack of progression of PERG amplitude reduction suggests the SO itself may affect the light stimulation and PERG signal or the limitations of detection by PERG. Nevertheless, these results suggest that RGCs are very sensitive to IOP elevation, but resilient for a period of time before further degeneration. Taken together, these in vivo results show that SOHU eyes developed progressive structural and visual function deficits that closely resemble changes in glaucoma patients.

Glaucomatous Degeneration of RGC Somata and Axons in SOHU Eyes

In vivo functional and imaging results indicate significant neurodegeneration in SOHU eyes, and histological analysis of post-mortem tissue samples supports these findings. The surviving RGC somata in retinal wholemounts and surviving axons in ON semithin cross-sections at multiple time points after SO injection were quantified. Similar to the changes of GCC thickness measured by

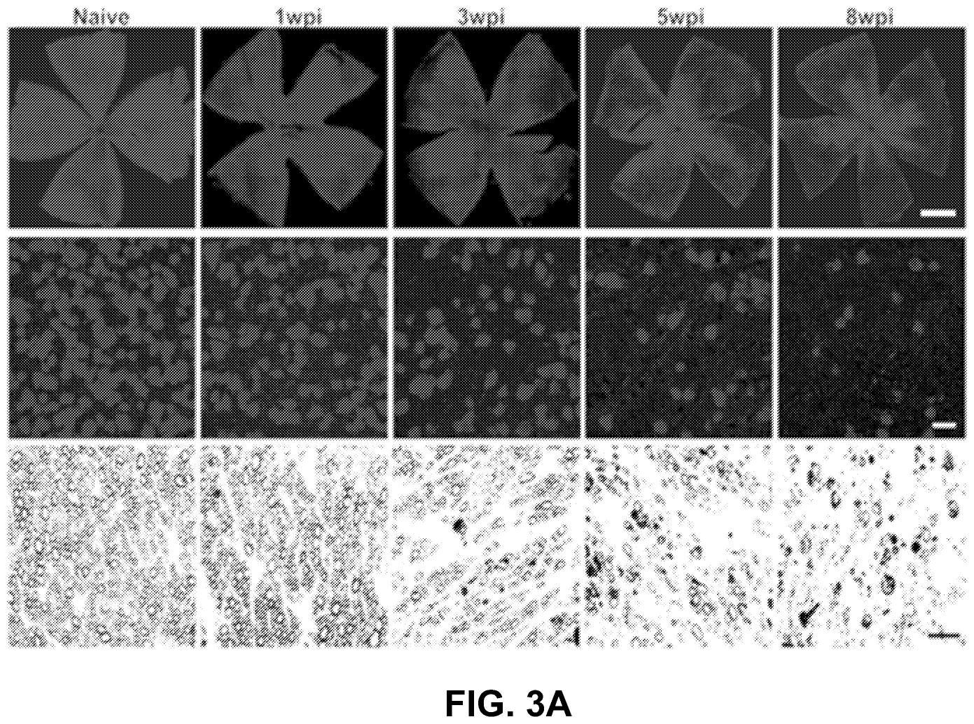

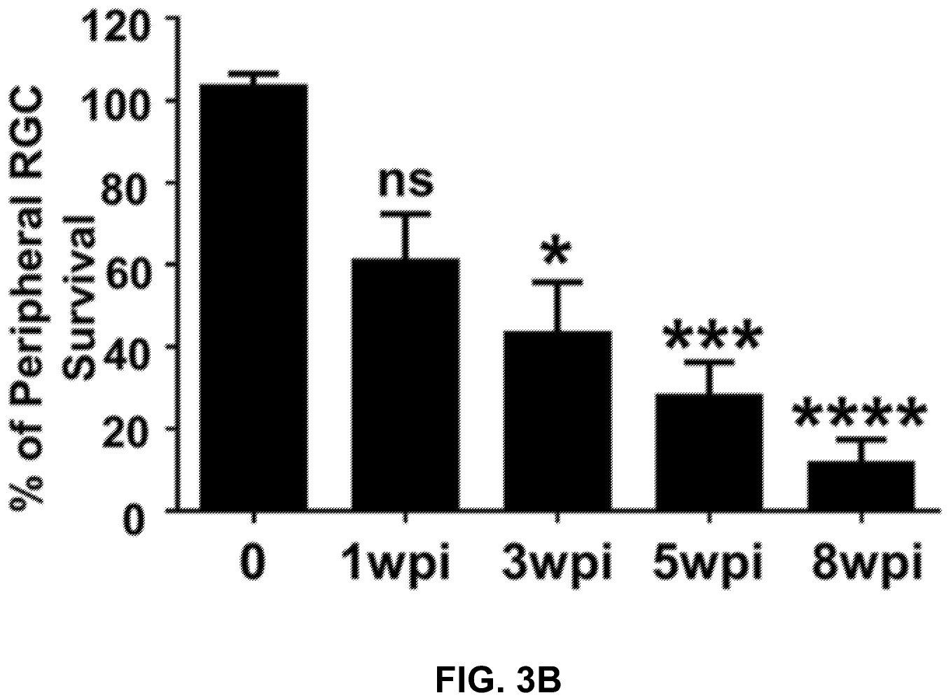

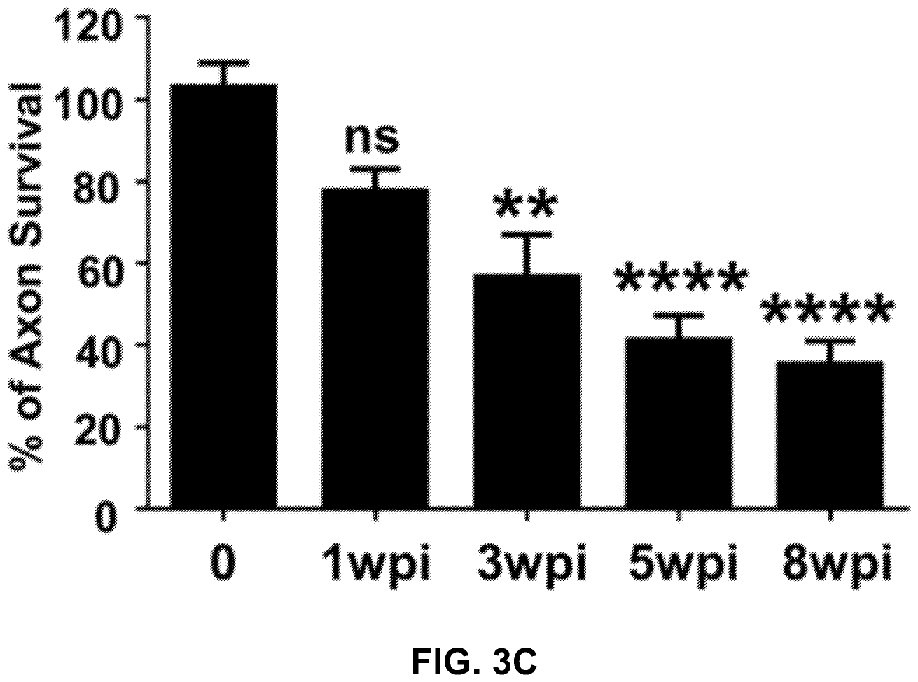

OCT in vivo, there was no statistical significance in surviving RGC counts in the peripheral retina between SOHU and control eyes at lwpi, whereas there was significant and worsening RGC loss at 3, 5 and 8wpi, when only 43, 28, and 12% of peripheral RGCs survived (FIGS. 3A, B). This result confirmed significant progressive RGC death in response to TOP elevation in SOHU eyes. io Significant RGC axon degeneration also occurred in SOHU ONs; only 57, 41% and 35% RGC axons survived at 3, 5, and 8wpi (FIGS. 3A, B). Therefore, TOP elevation in SOHU mouse eyes produces glaucomatous RGC and ON degeneration that starts as early as 3wpi and becomes progressing more severe at later time points that correlate with visual function deficits.

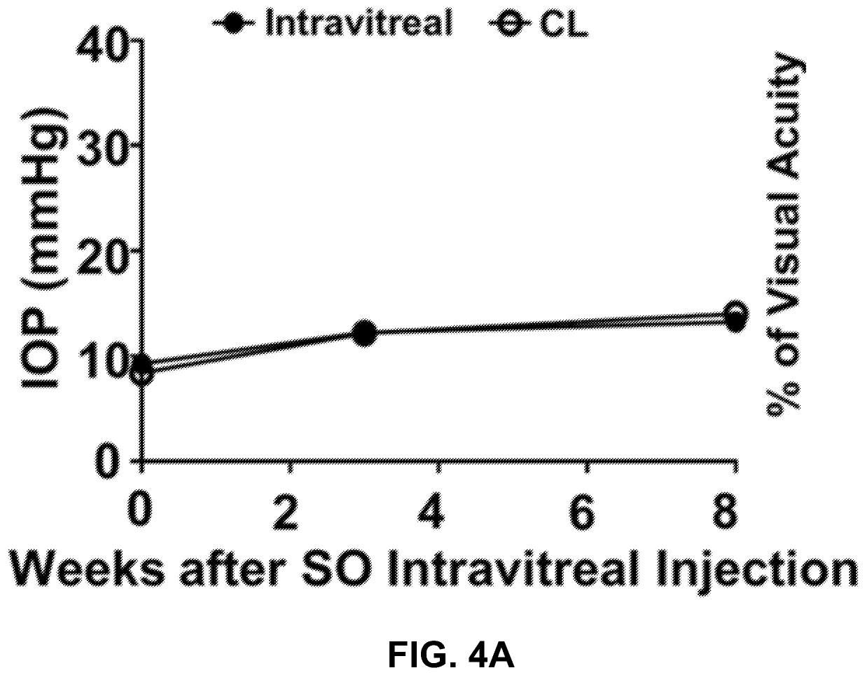

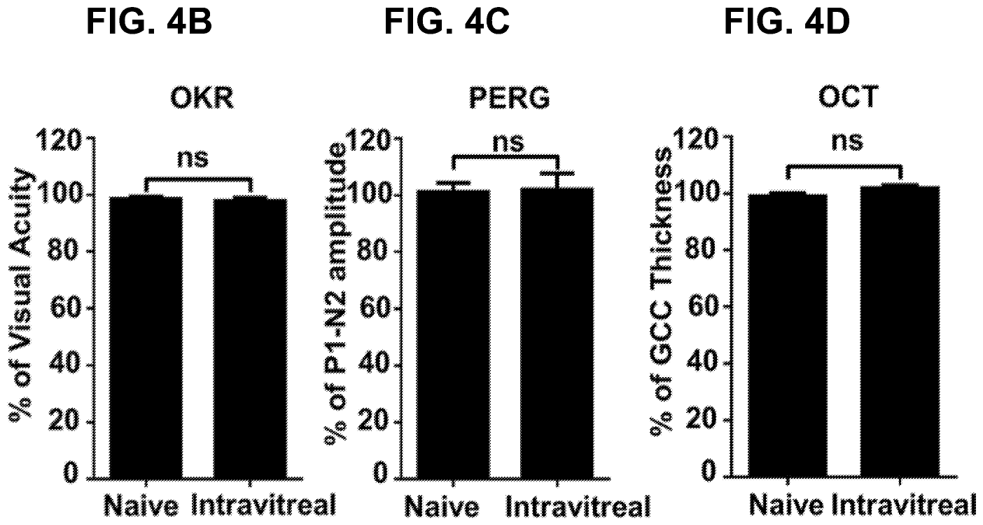

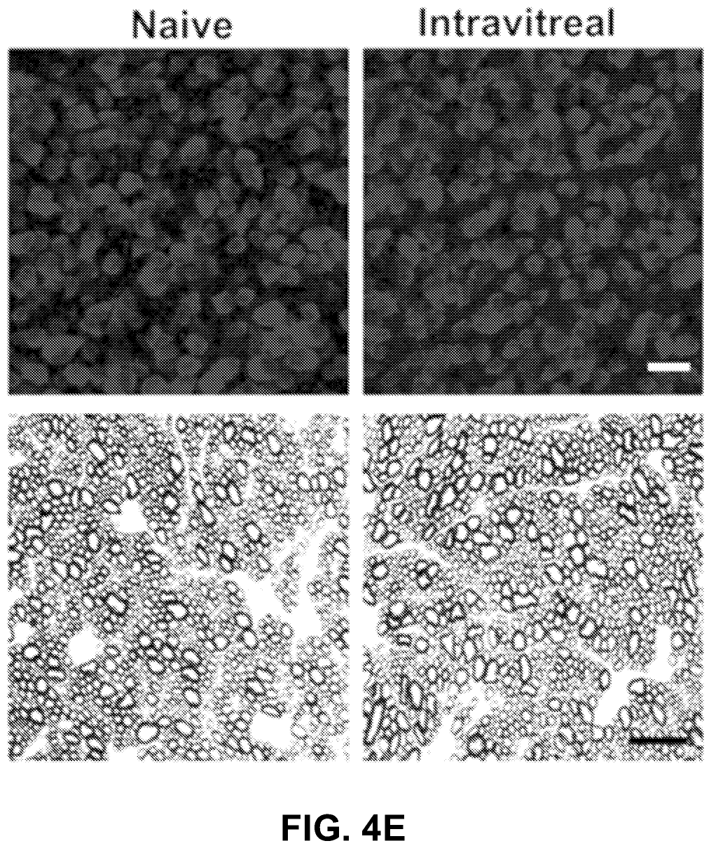

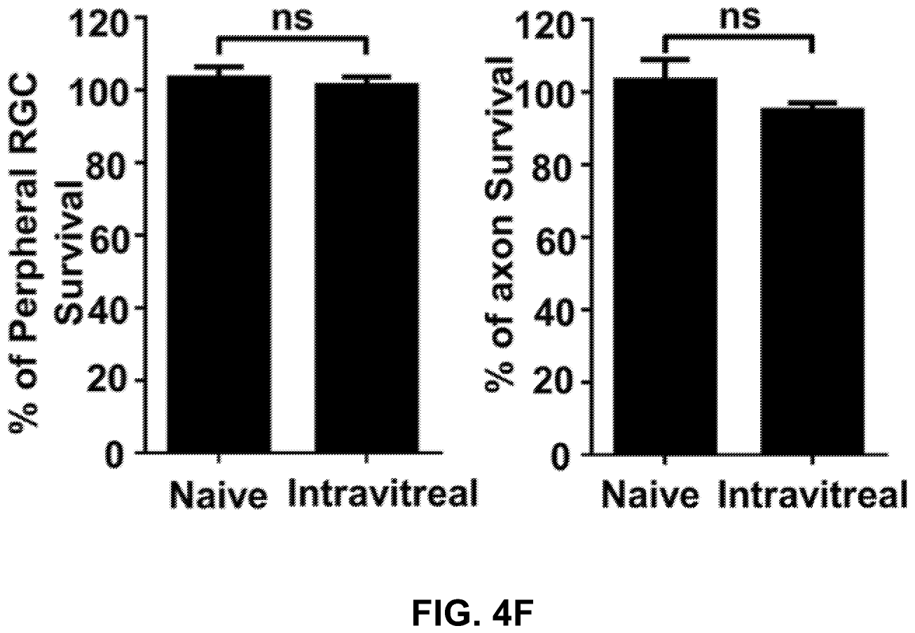

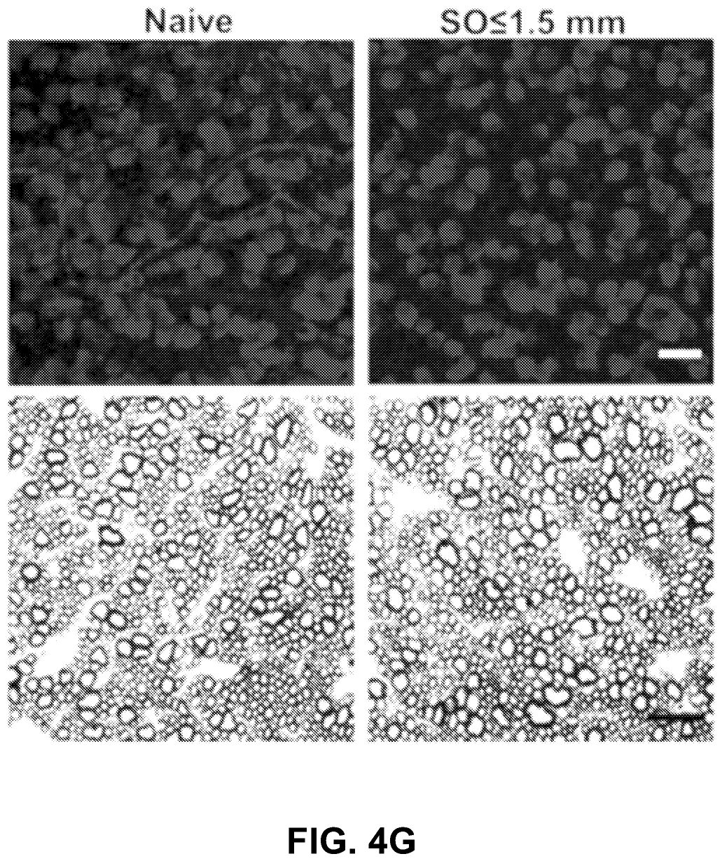

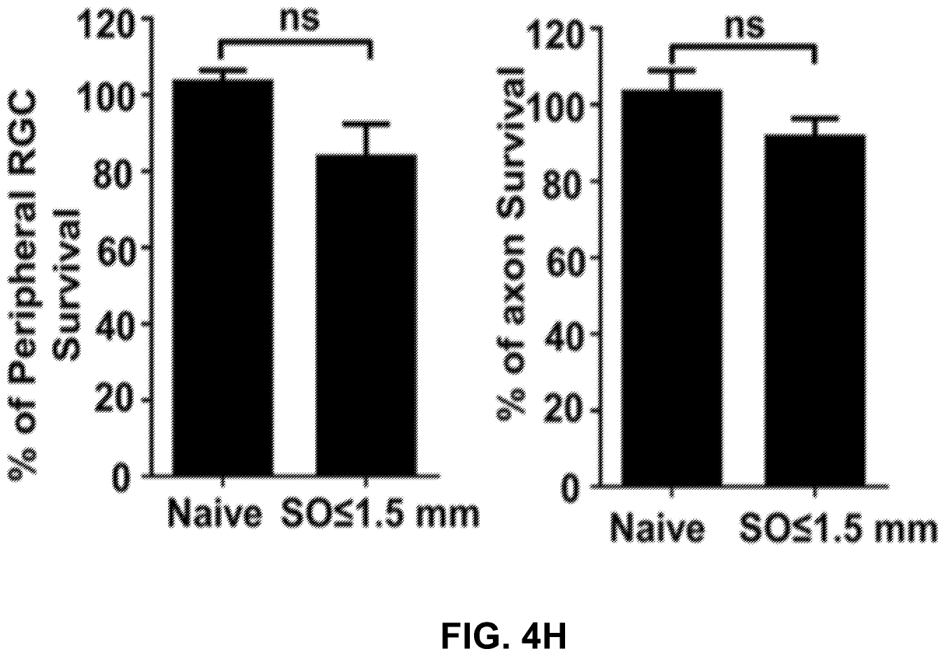

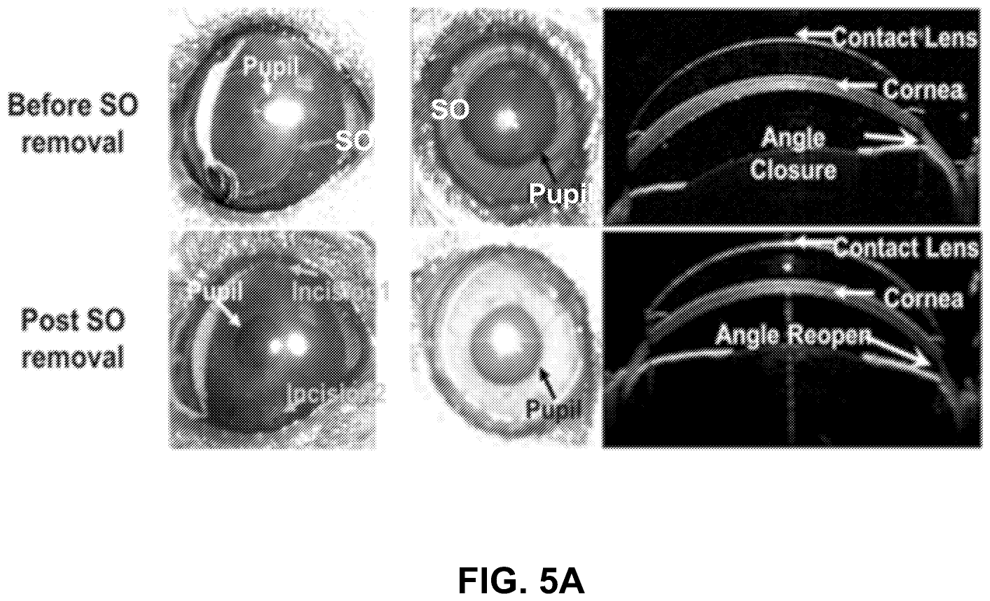

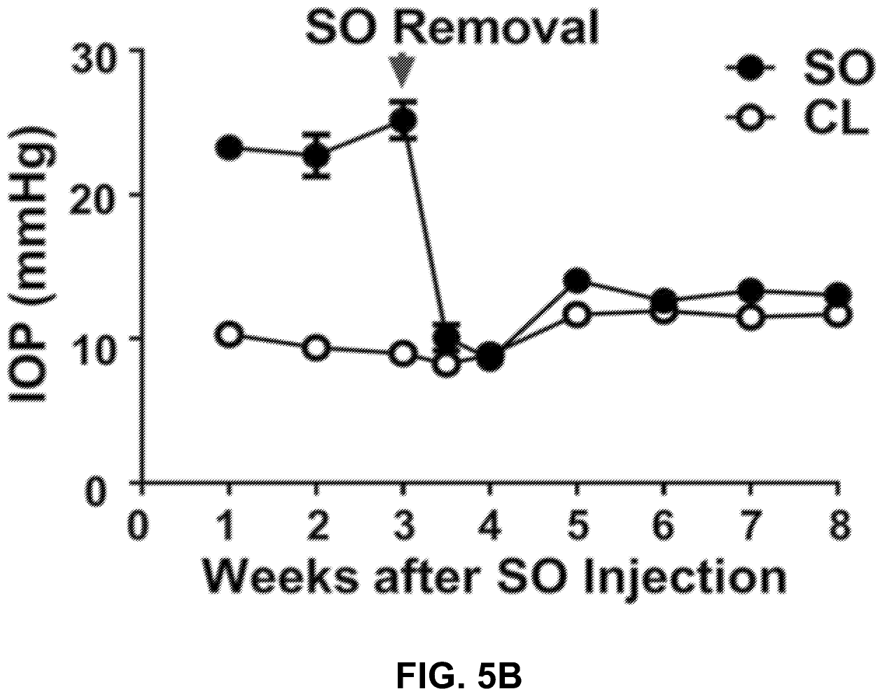

is Although the SO used in these studies was sterile and safe for human use, it was considered that toxicity might play a role in RGC death. Two experiments, however, provided evidence against this possibility: First, SO intravitreal injection did not cause significant TOP elevation, visual function deficits, or RGC/ON degeneration at 8wpi (FIGS. 4A-F). Second, the eyes with small SO droplets (≤1.5 mm) and unstable TOP elevation (FIG. 1D) showed no significant RGC death or axon degeneration at 8wpi (FIGS. 4G, H). Therefore, it was concluded that the neurodegeneration phenotypes observed in SOHU eyes are glaucomatous responses to ocular hypertension.

SOHU is a reversible ocular hypertension model

One of the disadvantages of other glaucoma models is that the initial eye injury is irreversible. However, with the model and methodology of this invention, the inventors were able to flush out the SO from the anterior chamber with the aid of normal saline infiltration (FIG. 5A). This procedure lowered the TOP back to normal quickly and stably (FIG. 5B), suggesting that SOHU is a reversible model that can be used to test whether lowering TOP affects degeneration of glaucomatous RGCs or the combination effect with neuroprotection.

What is claimed is:

1. A silicone oil-induced ocular hypertension glaucoma model, comprising: an experimental eye with an anterior chamber having in the anterior chamber a silicone oil droplet larger than 1.5 mm in diameter.

2. The silicone oil-induced ocular hypertension glaucoma model as set forth in claim 1, wherein the silicone oil droplet is equivalent to about 1-2 microliters.

3. The silicone oil-induced ocular hypertension glaucoma model as set forth in claim 1, further comprising a contralateral eye with an anterior chamber having in the anterior chamber a volume of saline which is used as a control eye relative to the experimental eye.

4. The silicone oil-induced ocular hypertension glaucoma model as set forth in claim 3, wherein the volume of saline is equivalent to about 1-2 microliters.

5. A method of modeling intraocular hypertension, comprising: injecting into an anterior chamber of an experimental eye a silicone oil to form a droplet of at least 1.5 mm in diameter inside the anterior chamber.

6. The method as set forth in claim 5, wherein the injected silicone oil is equivalent to about 1-2 microliters.

7. The method as set forth in claim 5, further comprising injecting into an anterior chamber of a contralateral eye a volume of saline which is used as a control eye relative to the experimental eye.

8. The method as set forth in claim 7, wherein the volume of saline is equivalent to io about 1-2 microliters.
